(19) United States
(12) Patent Application Publication
(10) Pub. No.: US 2024/0293548 A1
(43) Pub. Date: Sep. 5, 2024

(54) CONTACT LENS FOR PHOTODYNAMIC INACTIVATION OF GERMS, PRODUCT AND METHOD OF TREATING FUNGAL KERATITIS BY APPLYING THE SAME

(71) Applicant: NATIONAL CHENG KUNG UNIVERSITY, Tainan City (TW)

(72) Inventors: Tak-Wah WONG, Tainan City (TW); Jia-Horung HUNG, Tainan City (TW)

(21) Appl. No.: 18/506,757

(22) Filed: Nov. 10, 2023

(30) Foreign Application Priority Data
Mar. 1, 2023 (TW) 112107385

(51) Int. Cl. A61K 41/00; A45C 11/00; A61K 9/00; A61K 33/40; A61L 12/06; A61P 31/10

(52) U.S. Cl. CPC A61K 41/008; A45C 11/005; A61K 9/0051; A61K 33/40; A61L 12/06; A61P 31/10

(57) ABSTRACT

The present invention is of using contact lenses for photodynamic inactivation of germs, a product, and a method of treating fungal keratitis by applying the same. The contact lens is to continuously release a photoactive solution containing a photosensitizer such as rose bengal and hydrogen peroxide to the ocular surface. The photosensitizer would be activated while these contact lenses are applied and exposed to daylight or other artificial lights in the environment. After activation, the photosensitizer will produce singlet oxygen and reactive oxygen species to inhibit the growth of fungi, thereby treating fungal keratitis without having the patient experiences eye pain or discomfort. Moreover, since the photoactive solution excludes any antifungal agents, the contact lenses for photodynamic inactivation of germs of the present invention can not only improve drug-resistant fungal keratitis but also prevent the germs from developing antimicrobial resistance.

This application claims priority to Taiwan Application Serial Number TW112107385, filed Mar. 1, 2023, which is herein incorporated by reference in its entirety.

### BACKGROUND

### Field of Invention

The present invention relates to a contact lens. More particularly, the present invention relates to a contact lens for photodynamic inactivation of germs, a product, and a method of treating fungal keratitis by applying the same.

### Description of Related Art

The cornea lies in the outermost of the anterior eyeball and is the first structure encountered by the light, and the cornea refracts the light while the light travels through it. Thus, corneal damage will affect vision and even cause blindness. According to global statistics, cornea diseases are the fourth leading cause of blindness, and infectious keratitis is one of the most common blinding cornea diseases.

Infectious keratitis is an infection of the cornea caused by germs such as viruses, bacteria, fungi, and parasites. Among the infectious keratitis, fungal keratitis caused by fungi such as Candida spp., Fusarium spp., and Aspergillus spp. is more common in Asia. Conventional methods for treating fungal keratitis include administering antifungal agents. However, there are only limited choices of antifungal agents. Moreover, the increased incidence of fungal keratitis caused by drug-resistant strains recently resulted in a lower effectiveness of the antifungal agents on fungal keratitis. The contemporary method of treating the infection caused by multiple drug-resistant strains is to perform a corneal transplantation surgery. However, after the surgery, the patients need to receive long-term topical or systemic immunosuppressant drugs to avoid rejections of the transplant, therefore increasing the risk of the recurrence of fungal keratitis.

Existing literature and studies have found that photodynamic therapy (abbreviated as PDT) can be used to inhibit the growth and activity of germs. In detail, after the photosensitizer is excited by a specific wavelength of light, singlet oxygen, and reactive oxygen species (abbreviated as ROS) are generated during PDT which can inhibit the growth and activity of germs. PDT with high light power can shorten the treatment duration, but it can also cause eye discomfort such as pain, irritation, and photophobia, and even require anesthesia before and during the procedure to relieve the discomfort. PDT with low light power (which can be performed with natural daylight and artificial lights such as a fluorescent lamp) may not cause significant eye discomfort but requires longer treatment duration. A specific amount of photosensitizer is required to keep on the ocular surface during PDT with low or high light power. However, the photosensitizer may be removed by the blinking and degraded by enzymes in the tear, making it hard to keep on the surface of the eye. Thus, the contemporary approach requires frequent application of the photosensitizer drops (e.g., every five minutes), and the eyes are kept open with eyelid speculums so that blinking is avoided. This is to keep the photosensitizer on the surface of the eye, so the concentration in the corneal stroma would be adequate.

Given the above, it is necessary to establish a continuously-releasing method of the photosensitizer to the ocular surface, with important modification of the photosensitizers to enhance the germ-killing effect, especially for PDT in low light power.

### SUMMARY

Thus, the present invention provides contact lenses that are loaded with specially designed photosensitizers for the photodynamic inactivation of germs to solve the aforementioned problems.

One aspect of the present invention is to provide a contact lens for the photodynamic inactivation of germs. By using the contact lens, the photoactive solution can be continuously released to the ocular surface. The photoactive solution includes rose bengal (RB) and hydrogen peroxide (H₂O₂). The photoactive solution continuously released by the contact lens can produce singlet oxygen and reactive oxygen species, which can effectively inhibit the growth and activity of the fungi, thereby improving fungal keratitis. This method would not cause discomforts such as fatigue, pain, and irritation after exposure to white light for a long duration. Moreover, since the photoactive solution excludes antifungal agents, the application of the contact lens for photodynamic inactivation of germs of the present invention can not only improve fungal keratitis caused by drug-resistant strains but can also prevent the germs from developing antimicrobial resistance.

Another aspect of the present invention is to provide a product of a contact lens for the photodynamic inactivation of germs, which includes a packaging structure, a photoactive solution accommodated in the packaging structure, and the contact lens for photodynamic inactivation of germs submerged in the photoactive solution.

The other aspect of the present invention is to provide a method of treating fungal keratitis with the contact lens with the mechanism of photodynamic inactivation of germs. By applying the aforementioned contact lens that has been submerged in the aforementioned photoactive solution on an eye with an infected cornea, followed by exposing the infected eye to white light, the photoactive solution continuously released from the contact lens can inhibit the growth and activity of germs.

According to the aforementioned aspect, the invention provides a contact lens for the photodynamic inactivation of germs, which includes a contact lens and a photoactive solution absorbed by the contact lens. The material of the contact lens is a hydrogel or a silicone hydrogel. The photoactive solution includes 0.01 wt % to 1.0 wt % rose bengal, 0.01 wt % to 1.0 wt % hydrogen peroxide, and a buffer solution to balance the pH and osmolality but excludes antifungal agents. After the contact lens is exposed to white light for 0.01 hour to 16 hours, the photoactive solution continuously released by the contact lens produces singlet oxygen and reactive oxygen species, thereby inhibiting growth and activity of fungi.

In one embodiment of the invention, the buffer solution includes one or more of the following buffers: tris(hydroxymethyl)aminomethane (abbreviated as Tris) buffer, 4-(2-hydroxyethyl) piperazine-1-ethane sulfonic acid hemisodium salt (abbreviated as HEPES) buffer, phosphate buffered saline (abbreviated as PBS), and glycylglycine buffer.

In one embodiment of the invention, the photoactive solution further includes a stabilizer.

In one embodiment of the invention, the white light has a light intensity greater than or equal to 0.1 mW/cm² at its 520 nm wavelength.

In one embodiment of the invention, the white light contains green light with a light dose of 0.01 J/cm² to 200 J/cm² and a wavelength of 495 nm to 570 nm.

In one embodiment of the invention, the fungi are drug-resistant strains.

In one embodiment of the invention, the fungi are Candida spp., Fusarium spp., and/or Aspergillus spp.

According to another aspect, the invention provides a product of the contact lens for the photodynamic inactivation of germs. The product includes a packaging structure, a cover sheet, a photoactive solution, and a contact lens. The packaging structure includes an accommodating portion having a groove portion and a flat portion surrounding the groove portion. The cover sheet is removably attached to the accommodating portion to disclose the groove portion. The photoactive solution is accommodated in the groove portion, the photoactive solution contains 0.01 wt % to 1.0 wt % rose bengal, 0.01 wt % to 1.0 wt % hydrogen peroxide and a buffered solution with a balance, and the photoactive solution excludes antifungal agents. The contact lens is accommodated in the groove portion and submerged in the photoactive solution, and the contact lens is a hydrogel or a silicone hydrogel. After the contact lens is exposed to white light for 0.01 hour to 16 hours, the photoactive solution continuously released by the contact lens produces singlet oxygen and reactive oxygen species, thereby inhibiting growth and activity of fungi.

In one embodiment of the invention, the accommodating portion is opaque.

According to the other aspect, the present invention provides a method of treating fungal keratitis by applying a contact lens for the photodynamic inactivation of germs. First, a contact lens is submerged in a photoactive solution, the contact lens is a hydrogel or a silicone hydrogel, and the photoactive solution contains 0.01 wt % to 1.0 wt % rose bengal, 0.01 wt % to 1.0 wt % hydrogen peroxide, and a buffer solution with a balance. Then, the contact lens is applied to an eye with an infected cornea. Next, the infected eye being covered with the contact lens is exposed to white light for 0.01 hour to 16 hours. The photoactive solution continuously released by the contact lens produces singlet oxygen and reactive oxygen species, thereby inhibiting the growth and activity of fungi, and the photoactive solution excludes antifungal agents.

By applying the contact lens for photodynamic inactivation of germs, the product, and method of treating fungal keratitis by applying the same of the present invention, the contact lens made by hydrogel or a silicone hydrogel can continuously release photoactive solution including rose bengal and hydrogen peroxide to the ocular surface. After the contact lens is exposed to white light for a long duration, the photoactive solution continuously released from the contact lens produces singlet oxygen and reactive oxygen species, which can inactivate the growth and activity of fungi, thereby improving fungal keratitis. Since the light dose requirement is low, it will not cause eye discomfort such as pain and irritation. In addition, the photoactive solution excludes antifungal agents, the contact lens for photodynamic inactivation of germs of the present invention can not only improve fungal keratitis caused by drug-resistant strains but can also prevent the germs from developing antimicrobial resistance.

### BRIEF DESCRIPTION OF THE DRAWINGS

The invention can be more fully understood by reading the following detailed description of the embodiment, with reference made to the accompanying drawings as follows:

FIG. 1 is an exploded view diagram of a product 100 of the contact lens for photodynamic inactivation of germs according to some embodiments of the present invention.

FIGS. 2A and 2B are bar charts showing the amounts of C. albicans treated with 0.2 wt % rose bengal and exposed to light (10 J/cm² in FIG. 2A and 20 J/cm² in FIG. 2B, respectively) according to an embodiment of the present invention.

FIG. 3 is a bar chart showing the amount of C. albicans treated with different concentrations of hydrogen peroxide according to some embodiments of the present invention.

FIGS. 4A and 4B are curve graphs illustrating the 24-hour cumulative release of RB of the submerging group after the contact lenses were submerged in the 0.01 wt % RB solution, and 0.1 wt % RB solution and that of the dripping group of a 0.01 wt % RB solution, and 0.1 wt % RB solution was applied with drops, respectively, according to some embodiments of the present invention.

FIGS. 5A and 5B illustrated curve graphs of the at least 300-hour (about 2-week) cumulative release of RB from the contact lenses of the submerging group submerged in the 0.01 wt % RB solution (FIG. 5A) and 0.1 wt % RB solution (FIG. 5B) and that of the dripping group that was applied with drops of the 0.01 wt % RB solution (FIG. 5A) and 0.1 wt % RB solution (FIG. 5B), respectively, according to some embodiments of the present invention.

FIG. 6 is a bar chart illustrating the amount of C. albicans in the fungal solution of C. albicans after different treatments according to some embodiments of the present invention.

### DETAILED DESCRIPTION

As mentioned above, the present invention provides a contact lens for the photodynamic inactivation of germs, a product, and a method of treating fungal keratitis by applying the same. Since the contact lenses can continuously release photoactive solution to the ocular surface, the fungal keratitis can be effectively improved as the patient wears the contact lenses for photodynamic inactivation of germs and activates under natural daylight or normal indoor light without causing discomforts such as pain and irritation.

The aforementioned contact lens for photodynamic inactivation of germs can include a contact lens and photoactive solution. The contact lens can be a soft contact lens whose material can be a hydrogel or a silicone hydrogel, for example to absorb the photoactive solution.

The photoactive solution can include but is not limited to a photosensitizer and hydrogen peroxide. There is no specific limitation on the kind of photosensitizer except that the photosensitizer could be activated by visible light. In some specific embodiments, the photosensitizer can include but is not limited to toluidine blue solution, methylene blue, rose bengal (abbreviated as RB), indocyanine green (abbreviated as ICG), aminolevulinic acid, and vitamin B2. The amount of photosensitizer in the photoactive solution depends on the kind of photosensitizer. For example, when the photosensitizer is RB, the photoactive solution can contain 0.01 wt % to 1.0 wt % RB. If the photoactive solution contains too little amount of RB, the antimicrobial efficacy of the contact lens for the photodynamic inactivation of germs is less effective. If the photoactive solution contains excessive RB, the photoactive solution has the risk of causing ocular pain and irritation.

The concentration of hydrogen peroxide in the photoactive solution is within the safe dose since hydrogen peroxide is a strong oxidant that will cause discomfort such as pain and irritation on human skin and mucous membranes, including ocular surface. Generally, the concentration of hydrogen peroxide used as a skin disinfectant or antimicrobial agent for contact lenses cleansing solution is 3 weight (wt) %. However, hydrogen peroxide in this amount can still damage the ocular surface, that is, chemical burn to the corneal and conjunctival epithelium. The concentration of hydrogen peroxide in the aforementioned photoactive solution can be 0.01 wt % to 1.0 wt %, for example, which is much lower than the concentration of hydrogen peroxide for skin disinfectant. If the concentration of hydrogen peroxide is too low, the contact lens for photodynamic inactivation of germs may not inactivate germs effectively under the white light of a low light dose. If the concentration of hydrogen peroxide is too high, the photoactive solution harms the eyes instead.

The hydrogen peroxide can be prepared by commercial hydrogen peroxide products that generally contain 35 wt % hydrogen peroxide. Since hydrogen peroxide is a strong oxidant and can be degraded after exposure to air, commercial hydrogen peroxide products can selectively contain a stabilizer. In some embodiments, based on 100 wt % of the hydrogen peroxide products, the hydrogen peroxide products can selectively contain 0.05 wt % to 1 wt % stabilizer to enhance the stabilization of the hydrogen peroxide. The kind of stabilizer is not specifically limited and can be selected from the group consisting of acetaminophen, acetanilide, phenacetin, polyvinyl alcohol (abbreviated as PVA), and any combination thereof or any other stabilizer contained in the commercial hydrogen peroxide products for wound disinfection. The concentration of hydrogen peroxide in the photoactive solution is low, and thus the photoactive solution can selectively contain the aforementioned stabilizer or not.

The buffer solution is used as the solvent for the photosensitizer and the hydrogen peroxide. The kinds of buffer solution have no specific limitations, but it will be better if the buffer solution can be used on the eyes. In one specific embodiment, the buffer solution can include but is not limited to tris(hydroxymethyl)aminomethane (abbreviated as Tris) buffer, 4-(2-hydroxyethyl) piperazine-1-ethane sulfonic acid hemisodium salt (abbreviated as HEPES) buffer, phosphate buffered saline (abbreviated as PBS) buffer, and glycylglycine buffer.

The term “antimicrobial efficacy” herein referred to the effect of decreasing the germ amount, i.e., to inhibit the growth and activity of germs effectively or to inactivate germs. In some embodiments, achieving antimicrobial efficacy indicates meeting the Taiwan and USA disinfectant standards, i.e., the log reduction value of germs is more than or equal to 3 (more than or equal to 3-log reduction, equivalent to an antimicrobial rate more than or equal to 99.9%). In some embodiments, achieving antimicrobial efficacy indicates meeting the European Union (EU) disinfectant standard, i.e., the log reduction value of germs is more than or equal to 4 (more than or equal to 4-log reduction, equivalent to an antimicrobial rate more than or equal to 99.99%). The aforementioned antimicrobial rate indicates the percentage of the difference between the initial germ amount and the germ amount after the disinfection treatment to the initial germ amount.

There is no special limitation on the aforementioned germs. Those causing infectious keratitis are included. In some embodiment, the aforementioned germs can include but are not limited to bacteria, fungi, protozoa, and virus. In some embodiments, the germs can include Candida spp., Fusarium spp., and/or Aspergillus spp. In some specific embodiment, the germs can be C. albicans, C. krusei, C. glabrata, C. parapsilosis, C. tropicalis, C. lusitaniae, C. guilliermondii, C. dubliniensis, and/or C. auris. Among the germs, C. albicans is the most common. The strain of the germ is not limited and can be a common strain. In some embodiment, the germ can be a drug-resistant strain of C. albicans.

The term “photodynamic inactivation of germs” refers to achieving antimicrobial efficacy with a photoactive solution, light, and oxidation. In detail, the photosensitizer of the aforementioned photoactive solution can produce singlet oxygen and reactive oxygen species after the photosensitizer is exposed to light and forms activated photosensitizer, followed by further interaction with the oxygen molecules in the environment, thereby inhibiting the growth and activities of germs. The reactive oxygen species can include but are not limited to superoxide radicals and hydroxyl radicals.

The wavelength of the aforementioned light is selected based on the selection of the photosensitizer. As mentioned above, the photosensitizer that can be activated by visible light, such as light containing wavelengths from 400 nm to 760 nm, is selected. In some embodiments, the light is combined with two light colors (wavelength). In other embodiments, the light contains a continuous spectrum. In other specific embodiments, white light includes green light having wavelengths of 495 nm to 570 nm. In a specific example, white light includes green light having a wavelength of 520 nm when the photosensitizer is RB. The selection of light source of the aforementioned white light is not specifically limited and can be selected from natural daylight or artificial lighting sources, and the artificial lighting sources can include but are not limited to light-emitting diode (abbreviated as LED), fluorescent lamps, halogen lamps and incandescent lamps.

After the photoactive solution is exposed to light and activated, the obtained excited photosensitizer can interact with the oxygen molecules in the environment, thereby obtaining singlet oxygen and reactive oxygen species. Therefore, the dose of the light is determined by the activity of the activated photosensitizer to produce singlet oxygen and reactive oxygen species. The source of the oxygen molecules in the drug-administration environment is not specially limited. In some embodiments, the oxygen molecules in the environment can be originated from the biomolecules (e.g., lipid, protein and amino acid, etc.) of a germ or oxygen molecules in biological tissues. In some embodiments, the oxygen molecules in the environment can be originated from the hydrogen peroxide of a photoactive solution. In some embodiments, when the photosensitizer is RB, the white light contains green light with a wavelength of 495 nm to 570 nm, and the light dose of green light is 0.01 J/cm² to 200 J/cm² or 5 J/cm² to 35 J/cm² or 5 J/cm² to 20 J/cm².

It should be noted that when the photoactive solution includes a photosensitizer and hydrogen peroxide, antimicrobial efficacy can be achieved with a low light dose. In contrast, a higher light dose is required when the photoactive solution only contains a photosensitizer. In vitro experiments have proved that when the photoactive solution includes a photosensitizer and hydrogen peroxide, the log reduction value of germs can be greater than or equal to 6 (greater than or equal to 6-log reduction, equivalent to an antimicrobial rate higher or equal to 99.9999%) after the photoactive solution is exposed to light with a low light dose (e.g., less than or equal to 10 J/cm²), indicating that the photoactive solution can achieve the antimicrobial efficacy. In contrast, when the photoactive solution includes only a photosensitizer but not hydrogen peroxide, the log reduction value of germs is only about 1 (1-log reduction, equivalent to an antimicrobial rate of 90%) after the photoactive solution is exposed to light with a low light dose (e.g., less than or equal to 10 J/cm²), indicating that the photoactive solution cannot achieve the antimicrobial efficacy. Nevertheless, the log reduction value of germs can reach 6 (6-log reduction, equivalent to an antimicrobial rate of 99.9999%) after the photoactive solution contains only a photosensitizer but not hydrogen peroxide is exposed to a higher light dose (e.g., 20 J/cm²). It should be noted that although the light dose is 10 J/cm² and 20 J/cm² in the aforementioned in vitro experiment, the antimicrobial efficacy can be achieved by the photoactive solution exposed to green light with a light dose of 5.0 J/cm² to 5.4 J/cm² since eyes have lysozyme and mucus immune system.

A light dose is defined as the product of light intensity and an exposure time of light. The light intensity is not especially limited and can be equivalent to the intensity of a white light source in daily life. In some embodiments, the white light contains a wavelength of 520 nm with a light intensity higher than or equal to 0.1 mW/cm² to achieve the aforementioned light dose during the daytime (about 12 hours), thereby achieving antimicrobial efficacy. In some specific embodiments, the white light contains a wavelength of 520 nm with a light intensity higher than or equal to 0.5 mW/cm². The white light source in daily life has been mentioned above and will not be elaborated on herein.

The light intensity of light outdoors in the sun is equal to 10 mW/cm² to 40 mW/cm² at a wavelength of 520 nm, indicating that antimicrobial efficacy can be achieved after the patient wears the contact lens for photodynamic inactivation of germs and activates outdoors in the sun for 2 minutes to 35 minutes. Moreover, the light intensity of light in the shadow is equal to 1 mW/cm² to 10 mW/cm² at a wavelength of 520 nm, indicating that antimicrobial efficacy can be achieved after the patient wears the contact lens for photodynamic inactivation of germs and activates outdoors in the shadow for 8 minutes to 5 hours. Furthermore, the light intensity of a general fluorescent lamp is equal to 0.1 mW/cm² to 1 mW/cm² at a wavelength of 520 nm, indicating that antimicrobial efficacy can be achieved after the patient wears the contact lens for photodynamic inactivation of germs and activates in a well-lit room for 1 to 12 hours.

As mentioned above, to photodynamically inactivate germs in the white light in daily life requires exposure to white light for many hours, and thus the aforementioned contact lens for photodynamic inactivation of germs is required to release the photoactive solution continuously during the period of the exposure of white light. As experiments have proved, by submerging the contact lens in the photoactive solution containing rose bengal, the obtained contact lens for photodynamic inactivation of germs can release rose bengal continuously for 300 hours.

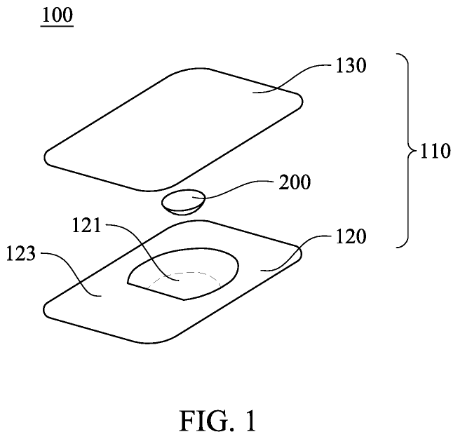

The aforementioned contact lens for photodynamic inactivation of germs can be packed in a packaging structure. Referred to FIG. 1, which is an exploded view diagram of a product 100 of the contact lens for photodynamic inactivation of germs according to some embodiments of the present invention. As shown in FIG. 1, the product 100 of the contact lens for photodynamic inactivation of germs contains a packaging structure 110 and a contact lens 200. In detail, the packaging structure 110 can contain an accommodating portion 120 and a cover sheet 130. The accommodating portion 120 includes a groove portion 121 and a flat portion 123 surrounding the groove portion, and the cover sheet 130 is removably attached to the flat portion 123 and discloses the groove portion 121. The contact lens 200 is accommodated in the groove portion 121.

In some embodiments, the product 100 of the contact lens for photodynamic inactivation of germs can selectively include a photoactive solution kit (not shown) containing the photoactive solution in a form of a concentrated solution or dry powder. In this embodiment, the concentrated solution or dry powder of the photoactive solution is diluted and added in the groove portion 121, and then the contact lens 200 is submerged in the photoactive solution for a period (e.g., 1 hour to 24 hours) before wearing. In the other embodiments, the groove portion 121 can selectively accommodate the photoactive solution, so that the contact lens 200 is submerged in the photoactive solution before the cover sheet 130 is removed from the flat portion 123. In the aforementioned embodiments, the accommodating portion 120 is opaque to prevent the photoactive solution from degrading under light exposure.

It is worth noting that as previous studies have shown, after the drug-resistant strains are subjected to photodynamic inactivation of germs, the surviving cells are more sensitive to antibiotics. Thus, the application of the contact lens for photodynamic inactivation of germs, which uses a contact lens to continuously release photoactive solution excluding an antifungal agent, can not only improve fungal keratitis caused by drug-resistant strains, but also prevent the germs from developing antimicrobial resistance, and therefore has the potential to be applied to improve the corneal infection caused by multidrug-resistant strains.

Although the present invention has been described in considerable detail regarding certain embodiments thereof, other embodiments are possible. Therefore, the spirit and scope of the appended claims should not be limited to the description of the embodiments contained herein.

### Example 1. Detecting Light Intensity of White Light in a Living Environment

The light intensities at wavelengths of 520 nm and 668 nm in the sunlight or at shady places were respectively measured outdoors for three executive days by using an illuminometer (manufacture: Ophir-Optronics, LCC, Israel) on sunny afternoons. The results were recorded in Table 1.

TABLE 1

| | | | Light intensity (mW/cm²) | | |
|---|---|---|---|---|---|
| | Wavelength | | Feb. 17, | Feb. 18, | Feb. 19, |
| Time | (nm) | Site | 2022 | 2022 | 2022 |
| 13:00 | 520 | in the sunlight | 48.6 | 42.6 | 49.9 |
| | | at shady place | 5.2 | 5.4 | 6.4 |
| | 668 | in the sunlight | 32.7 | 28.5 | 26 |
| | | at shady place | 3.5 | 3.6 | 4.3 |
| 14:30 | 520 | in the sunlight | 23.3 | 27.5 | 27.3 |
| | | at shady place | 5 | 3.8 | 7.2 |
| | 668 | in the sunlight | 15.5 | 18.8 | 19.7 |
| | | at shady place | 3.3 | 3.3 | 4.9 |
| 16:30 | 520 | in the sunlight | 20.1 | 22.2 | 19.3 |
| | | at shady place | 1.7 | 1.8 | 2.9 |
| | 668 | in the sunlight | 12.9 | 14.6 | 10.7 |
| | | at shady place | 1.1 | 1.2 | 1.9 |

As Table 1 had shown, no matter in the sunlight or at shady places, the light intensity gradually decreased as time passed by in the afternoon. However, the light in the sunlight had a light intensity of 19.3 mW/cm² to 49.9 mW/cm² at the a wavelength of 520 nm, and the light at shady places had a light intensity of 1.7 mW/cm² to 7.2 mW/cm² at the wavelength of 520 nm.

In the room with a fluorescent lamp turning on, the light intensities at the wavelength of 520 nm and 668 nm were measured by using the power meter at the different levels to the surface of a table: on the surface, 60 cm above the surface, and 60 cm below the fluorescent lamp. The results were recorded in Table 2.

TABLE 2

| Wavelength | | Light intensity |
|---|---|---|
| (nm) | Location | (mW/cm²) |
| 520 | at the surface of a table | 0.2 |
| | 60 cm above the surface of a table | 0.5 |
| | 60 cm below the fluorescent lamp | 0.9 |
| 668 | at the surface of a table | 0.1 |
| | 60 cm above the surface of a table | 0.3 |
| | 60 cm below the fluorescent lamp | 0.6 |

As shown in Table 2, although the light intensity placed 60 cm below the fluorescent lamp was higher, the light in a well-lit room had a light intensity of 0.2 mW/cm² to 0.9 mW/cm² at the wavelength of 520 nm.

### Example 2. Determining Antimicrobial Efficacy of Rose Bengal-PDT

A drug-resistant strain of Candida albicans was bought from the Bioresource Collection and Research Center (BCRC), Food Industry Research and Development Institute (FIRDI), 331, Shipin Rd., East Dist., Hsinchu City, Taiwan (postcode: 300193) under the accession number of BCRC 21538. The same strain could also be bought from American Type Culture Collection (ATCC), VA, USA under an accession number of ATCC 10231.

The Candida albicans was grown and prepared in a fungal solution with an amount of 1×10⁷ CFU/mL and mixed with an equal volume of rose bengal (RB) solution (i.e., the mixed volume ratio was 1:1) to obtain a mixed solution containing 0.2 wt % RB (0.2 wt % RB group). The RB solution was prepared by mixing PBS and RB, and the mixed solution had an RB concentration of 0.2 wt %.

A portion of the mixed solution containing 0.2 wt % RB and fungi was exposed to green light of 10 J/cm² or 20 J/cm² at 25±1° C., and another portion of the mixed solution was not treated with light exposure. The light source of the aforementioned green light was a commercial green light LED with an emission peak at the wavelength of 520 nm. Then, the mixed solution was subjected to a serial dilution to obtain a first diluted solution (ten-fold dilution), a second diluted solution (100-fold dilution), and a third diluted solution (1000-fold dilution). Next, an agar culture plate was divided into 4 quadrants, with three drops (20 μL/drop) of the first diluted solution, three drops of the second diluted solution, and three drops of the third diluted solution put on different quadrants, respectively. Noted that three drops represented three repeats. Then, the agar culture plate was incubated at 37° C. for 24 hours. In the control group, the fungal solution was mixed with an equal volume of PBS. The colonies of the control group and the colonies from the drops of the mixed solution containing 0.2 wt % RB and the fungi (0.2 wt % RB group) on the agar culture plate were counted to calculate the amounts of C. albicans in each group.

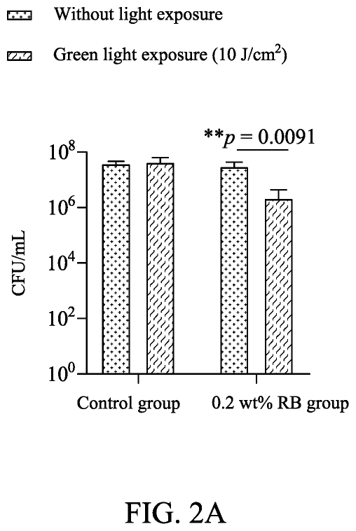

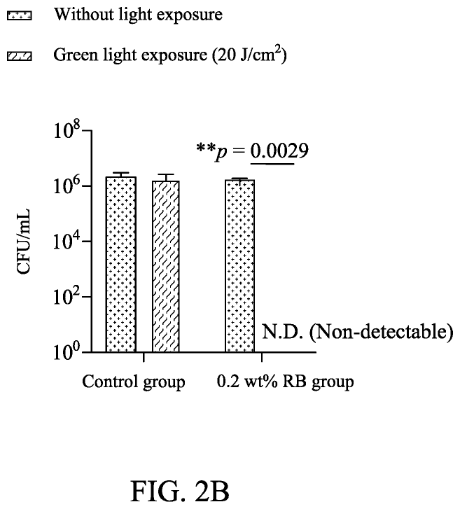

Referred to FIGS. 2A and 2B, which were bar charts showing the amounts of C. albicans treated with 0.2 wt % RB and exposed to light (10 J/cm² in FIG. 2A and 20 J/cm² in FIG. 2B) according to an embodiment of the present invention. In FIGS. 2A and 2B, the x-axes represented the control group and the 0.2 wt % RB group from left to right, the y-axes represented the amounts of C. albicans (unit: CFU/mL), the symbol “**” represented a statistically significant difference, and N.D. represented non-detectable.

As shown in FIG. 2A, in the control group, there was no statistically significant difference between the mixed solutions (i.e., the mixture of the fungal solution and PBS) exposed to 10 J/cm² green light and that without light exposure, indicating that the growth and activity of the C. albicans was not affected by green light. On the other hand, the amounts of C. albicans of the mixed solutions containing 0.2 wt % RB (i.e., the mixture of the fungal solution and RB solution) exposed to 10 J/cm² green light had a log reduction value of 1 (1-log reduction, equivalent to an antimicrobial rate of 90%) compared to that of without light exposure, indicating that the antimicrobial efficacy did not meet the requirement of a disinfectant, i.e., the log reduction value did not meet Taiwan and USA disinfectant standards (having a log reduction value more than or equal to 3, equivalent to an antimicrobial rate more than or equal to 99.9%) or EU disinfectant standards (having a log reduction value more than or equal to 4, equivalent to an antimicrobial rate more than or equal to 99.99%).

As shown in FIG. 2B, in the control group, there was no statistically significant difference between the mixed solutions (i.e., the mixture of the fungal solution and PBS) exposed to 20 J/cm² green light and those without light exposure. However, the amounts of C. albicans of the mixed solutions containing 0.2 wt % RB (i.e., the mixture of the fungal solution and RB solution) exposed to 20 J/cm² green light had a log reduction value of 6 (6-log reduction, equivalent to an antimicrobial rate of 99.9999%) compared to that of without light exposure, indicating that the mixed solution with 0.2 wt % RB required a 20 J/cm² green light exposure to achieve an effective antimicrobial efficacy.

### Example 3. Determining Rose Bengal Photodynamic Antimicrobial Efficacy after Adding Hydrogen Peroxide

1×10⁷ CFU/mL Candida albicans was grown in fungal solutions and were grouped into: (A) a negative control group, (B) a photodynamic treatment group, (C) a 0.1 wt % hydrogen peroxide with light exposure group, (D) a photodynamic treatment combining 0.1 wt % hydrogen peroxide group, and (E) photodynamic treatment combining 0.03 wt % hydrogen peroxide group. The fungal solution of the negative control group (A) contained neither RB nor hydrogen peroxide and was not exposed to light. The fungal solution of the photodynamic treatment group (B) did not contain hydrogen peroxide but contained 0.2 wt % RB and was exposed to 10 J/cm² green light. The fungal solution in (C) did not contain RB but contained 0.1 wt % hydrogen peroxide and was exposed to 10 J/cm² green light. The fungal solution in (D) contained 0.2 wt % RB and 0.1 wt % hydrogen peroxide and was exposed to 10 J/cm² green light. The fungal solution in (E) contained 0.2 wt % RB and 0.03 wt % hydrogen peroxide and was exposed to 10 J/cm² green light.

After that, the fungal solutions of each group were subjected to 3 serial dilutions to obtain a first diluted solution (ten-fold dilution), a second diluted solution (100-fold dilution), and a third diluted solution (1000-fold dilution). Next, an agar culture plate was divided into 4 quadrants and three drops of the fungal solutions, three drops of the first diluted solution, three drops of the second diluted solution, and three drops of the third diluted solution were put on each quadrant, respectively. Then, the agar culture plate was incubated at 37° C. for 24 hours. The colonies of each group on the agar culture plate were counted to calculate the amounts of C. albicans in each group.

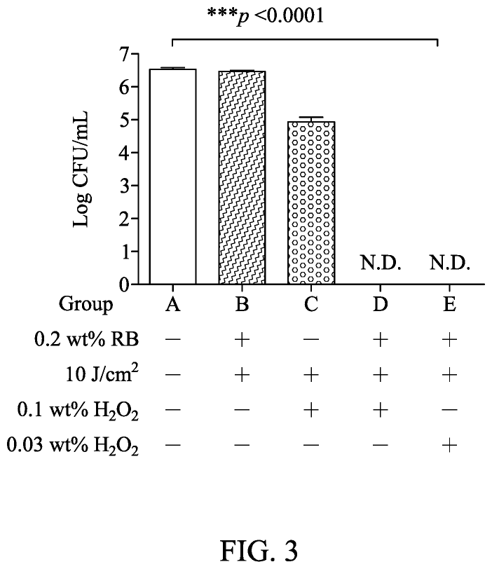

Referred to FIG. 3, which was a bar chart showing the amounts of C. albicans treated with different concentrations of hydrogen peroxide according to an embodiment of the present invention. In FIG. 3, the x-axis represented the negative control group (A), the photodynamic treatment group (B), the 0.1 wt % hydrogen peroxide with light exposure group (C), the photodynamic treatment combining 0.1 wt % hydrogen peroxide group (D), and the photodynamic treatment combining 0.03 wt % hydrogen peroxide group (E) from left to right, the y-axis represented the log values of the amounts of C. albicans (unit: log CFU/mL), the symbol “***” represented a statistically significant difference, and N.D. represented non-detectable.

As shown in FIG. 3, the log reduction values of the photodynamic treatment combining 0.1 wt % hydrogen peroxide group (D), and the photodynamic treatment combining 0.03 wt % hydrogen peroxide group (E) after being exposed to a low light dose (10 J/cm²) were at least 6 (at least 6-log reduction, i.e., an antimicrobial rate greater than or equal to 99.9999%). This indicated that the photoactive solution containing RB and 0.03 wt % to 0.1 wt % hydrogen peroxide could effectively enhance the antimicrobial efficacy after being exposed to green light of a low light dose (10 J/cm²).

### Example 4. Evaluating Whether Contact Lenses could Continuously Release the Photoactive Solution

First, RB solutions with different amounts of RB were prepared by methanol and RB, and the absorbance values at the wavelength of 548 nm were detected to establish a standard curve. Then, the contact lenses were divided into a submerging group and a dripping group. The contact lenses of the submerging group were submerged in a 0.01 wt % RB solution or a 0.1 wt % RB solution for 24 hours, respectively. In the dripping group, the contact lenses were applied with a drop of the 0.01 wt % RB solution or a drop of the 0.1 wt % RB solution every 5 minutes for 30 minutes, respectively.

The solution on the contact lens surfaces of the submerging and the dripping groups was removed by gently compressing the contact lenses with lens cleaning paper. Then, the contact lenses of the submerging group and the dripping group were respectively placed in wells of a 24-well plate containing 2 mL PBS, so that RB could be continuously released from the contact lenses. At each detecting time point, the solutions in the wells of the 24-well plate were detected by a spectrophotometer with absorbance values at a wavelength of 548 nm, and the contact lenses were placed in other wells containing fresh 2 mL PBS. The RB releasing amounts at each detecting time point were calculated corresponding to the standard curve. The curve of the cumulative release of RB can be obtained by adding up the RB releasing amounts at each detecting time point sequentially.

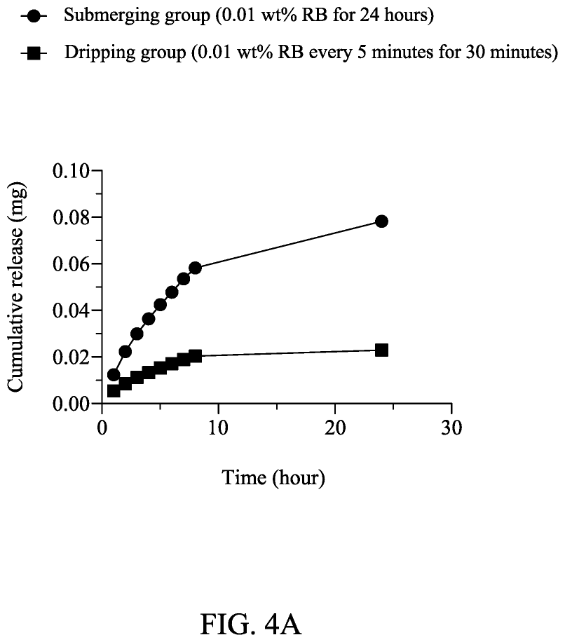

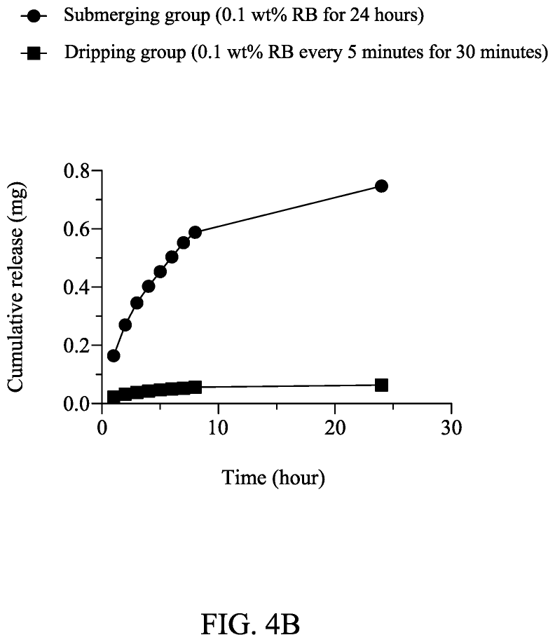

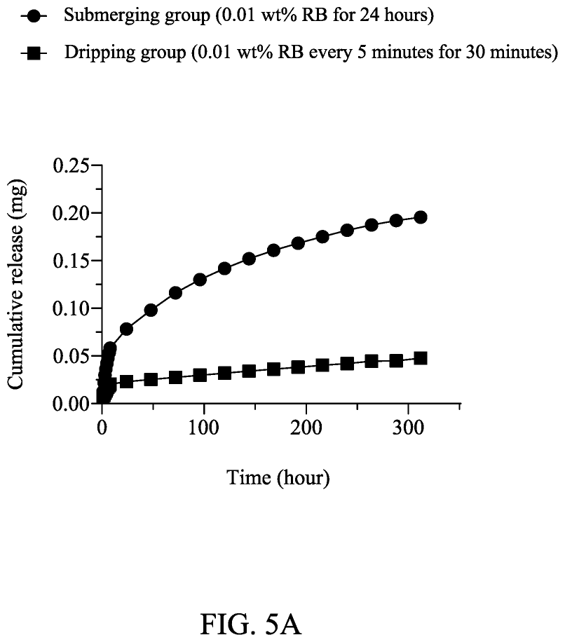

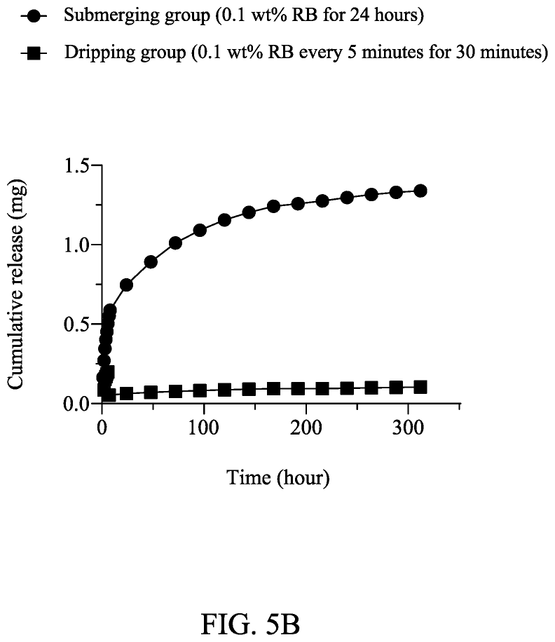

FIG. 4A and FIG. 4B were curve graphs illustrating the 24-hour cumulative release of RB of the submerging group after the contact lenses were submerged in the 0.01 wt % RB solution (FIG. 4A), and 0.1 wt % RB solution (FIG. 4B) and that of the dripping group of a 0.01 wt % RB solution (FIG. 4A) and 0.1 wt % RB solution (FIG. 4B) was applied with drops, respectively, according to some embodiments of the present invention. The x-axes represented time (unit: hour), and the y-axes represented the cumulative release of RB (unit: mg). FIG. 5A and FIG. 5B illustrated curve graphs of the at least 300-hour (about 2-week) cumulative release of RB from the contact lenses of the submerging group submerged in the 0.01 wt % RB solution (FIG. 5A) and 0.1 wt % RB solution (FIG. 5B) and that of the dripping group that was applied with drops of the 0.01 wt % RB solution (FIG. 5A) and 0.1 wt % RB solution (FIG. 5B), respectively, according to some embodiments of the present invention. The x-axes represented time (unit: hour), and the y-axes represented the cumulative release of RB (unit: mg).

As shown in FIG. 4A, FIG. 4B, FIG. 5A and FIG. 5B, either the curves of the contact lenses of the submerging group submerged in the 0.01 wt % RB solution or that submerged in the 0.1 wt % RB solution had a first slope in 8 hours and a second slope after 8 hours, and the first slope was greater than the second slope, indicating that the contact lenses could steadily release RB in 8 hours, and could continuously release RB at a lower rate after 8 hours. Moreover, the contact lenses of the submerging group could continuously release RB for at least 300 hours. In contrast, the dripping group simulated the clinical condition of applying a drop of RB solution every 5 minutes. The amount of RB released from the contact lenses of the dripping group was much less than that of the submerging group, indicating that it was difficult to keep the amount of the photoactive solution on the ocular surface by applying photoactive solution through dripping the RB on the cornea.

### Example 5. Determining the Antimicrobial Efficacy of the Photoactive Solution after Adding a Low Concentration of Hydrogen Peroxide

How the hydrogen peroxide affected the antimicrobial efficacy of the photoactive solution was further determined. The negative control (referred to as NC) group was the fungal solution of C. albicans not being treated with RB, hydrogen peroxide, and light exposure. The light control (referred to as LC) group was the fungal solution of C. albicans exposed to light but contained neither RB nor hydrogen peroxide. The dark control (referred to as DC) group was the fungal solution of C. albicans treated with 0.1 wt % RB for 15 minutes and did not expose to light. The hydrogen peroxide (referred to as H₂O₂) group was the fungal solution of C. albicans treated with 0.03 wt % hydrogen peroxide without RB for 15 minutes but not subjected to the light exposure treatment. The photodynamic therapy (referred to as PDT) group was the fungal solution of C. albicans treated with 0.1 wt % RB without hydrogen peroxide for 15 minutes and subjected to 35 J/cm² green light exposure treatment. The photodynamic therapy with 0.03 wt hydrogen peroxide (referred to as H₂O₂+PDT) group was the fungal solution of C. albicans treated with 0.1 wt % RB and 0.03 wt % hydrogen peroxide for 15 minutes and subjected to the 35 J/cm² green light exposure treatment.

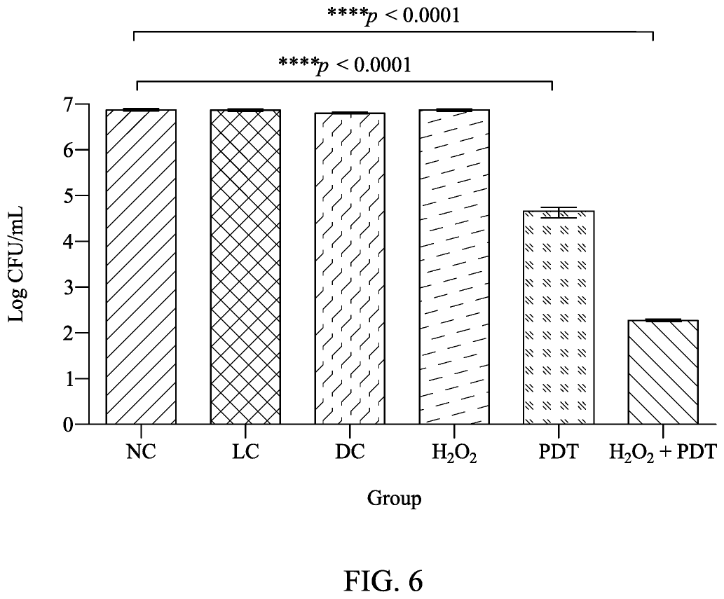

FIG. 6 was a bar chart illustrating the amount of C. albicans after different treatments in the fungal solution according to some embodiments of the present invention. The x-axis represented the NC group, the LC group, the DC group, the H₂O₂ group, the PDT group, and the H₂O₂+PDT group from left to right. The y-axis represented the C. albicans amount (unit: log CFU/mL). FIG. 6 showed the comprehensive results of 2 repeats in which n was 3, and the symbol “****” represented a statistically significant difference.

As shown in FIG. 6, there was no significant difference between the C. albicans amount of the NC group and those of the LC group, the DC group, and the H₂O₂ group. Noted that the concentration of hydrogen peroxide in the H₂O₂ group (0.03 wt %) was only 0.01 times the concentration of hydrogen peroxide in commercial hydrogen peroxide products for clinical disinfection of a wound, indicating that 0.03 wt % hydrogen peroxide alone could not achieve the antimicrobial efficacy indeed.

Moreover, compared to the NC group, the log reduction value of the C. albicans amount of the PDT group was 2.5 (2.5-log reduction, equivalent to an antimicrobial rate of 99.0% to 99.9%), indicating that the antimicrobial efficacy (i.e. log reduction value was more than or equal to 3) could not be achieved by treating C. albicans with 0.1 wt % RB and exposed to 35 J/cm² green light. However, compared to the NC group, the log reduction value of the C. albicans amount of the H₂O₂+PDT group was 5.5 (5.5-log reduction, equivalent to an antimicrobial rate of 99.999% to 99.9999%), indicating that the antimicrobial efficacy could be achieved by treating C. albicans with 0.1 wt % RB and 0.03 wt % hydrogen peroxide and exposed to 35 J/cm² green light, i.e. able to meet the Taiwan, USA and EU disinfectant standards. It was implied that a photoactive solution containing 0.1 wt % RB and 0.03 wt % hydrogen peroxide could achieve antimicrobial efficacy indeed after light exposure, and therefore could be applied to a contact lens for photodynamic inactivation of germs and the product of the same.

In sum, although a specific photoactive solution, a light source with a specific wavelength or a specific light dose, and specific evaluation methods are shown in the present invention as examples to explain the contact lens for photodynamic inactivation of germs, the product and the method of treating fungal keratitis by applying the same, it will be apparent to those skilled in the art that the present invention is not limited to what have mentioned. Without departing from the scope or spirit of the invention, it is intended that other photoactive solutions, light source with other wavelengths or other light doses, and other evaluation methods can also explain the present invention.

From the abovementioned embodiments, the contact lens for photodynamic inactivation of germs, the products, and the methods of treating fungal keratitis by applying the same have the advantages of continuously releasing the photoactive solution with the contact lens. Noted that the photoactive solution contains a photosensitizer and hydrogen peroxide, and thus the photoactive solution continuously released by the contact lens can produce reactive oxygen species and singlet oxygen after irradiation with a low light dose, thereby inhibiting the growth and activities of fungi. Therefore, a patient can improve his or her fungal keratitis in the meanwhile he or she wears the contact lens for photodynamic inactivation of germs and activated under natural daylight or normal indoor light without causing discomforts such as pain and irritation since the light dose required is low, and thus the light with a high light intensity is not required. Furthermore, eye pain is reduced because contact lens can act as a corneal band-aid, it can protect the damaged corneal epithelium from the mechanical shearing of the eyelids. In addition, the lens provides comfort without affecting the patient's vision. Moreover, since the photoactive solution excludes an antifungal agent, not only can the fungal keratitis caused by drug-resistant strains be improved by RB-PDT, but the germs can also be prevented from developing antimicrobial resistance.

It will be apparent to those skilled in the art that various modifications and variations can be made to the structure of the present invention without departing from the scope or spirit of the invention. Because of the foregoing, it is intended that the present invention cover modifications and variations of this invention provided they fall within the scope of the following claims.

What is claimed is:

1. A contact lens for photodynamic inactivation of germs, comprising:
a contact lens, wherein a material of the contact lens is a hydrogel or a silicone hydrogel; and
a photoactive solution, absorbed by the contact lens, wherein the photoactive solution comprises 0.01 wt % to 1.0 wt % rose bengal, 0.01 wt % to 1.0 wt % hydrogen peroxide, and a buffer solution with a balance, and the photoactive solution excludes an antifungal agent, and
after the contact lens is exposed to white light for 0.01 hour to 16 hours, the photoactive solution continuously released by the contact lens produces singlet oxygen and reactive oxygen species, thereby inhibiting growth and activity of fungi.

2. The contact lens for photodynamic inactivation of the germs of claim 1, wherein the buffer solution is selected from the group consisting of tris(hydroxymethyl)aminomethane (Tris) buffer, 4-(2-hydroxyethyl) piperazine-1-ethane sulfonic acid hemisodium salt (HEPES) buffer, phosphate-buffered saline (PBS), glycylglycine buffer, and any combination thereof.

3. The contact lens for photodynamic inactivation of the germs of claim 1, wherein the photoactive solution further comprises a stabilizer.

4. The contact lens for photodynamic inactivation of the germs of claim 1, wherein the white light has a light intensity greater than or equal to 0.1 mW/cm² at a wavelength of 520 nm.

5. The contact lens for photodynamic inactivation of the germs of claim 1, wherein the white light comprises green light with a light dose of 0.01 J/cm² to 200 J/cm², and a wavelength of the green light is 495 nm to 570 nm.

6. The contact lens for photodynamic inactivation of the germs of claim 1, wherein the fungi are drug-resistant strains.

7. The contact lens for photodynamic inactivation of the germs of claim 1, wherein the fungi are Candida spp., Fusarium spp., and/or Aspergillus spp.

8. A product of contact lens for photodynamic inactivation of germs, comprising:
a packaging structure, comprising:
an accommodating portion, comprising a groove portion and a flat portion surrounding the groove portion; and
a cover sheet, removably attached to the flat portion and disclosing the groove portion;
a photoactive solution, accommodated in the groove portion, wherein the photoactive solution comprises 0.01 wt % to 1.0 wt % rose bengal, 0.01 wt % to 1.0 wt % hydrogen peroxide, and a buffer solution with a balance, and the photoactive solution excludes an antifungal agent; and
a contact lens, accommodated in the groove portion and submerged in the photoactive solution, wherein the contact lens is a hydrogel or a silicone hydrogel, and
after the contact lens is exposed to white light for 0.01 hour to 16 hours, the photoactive solution continuously released by the contact lens produces singlet oxygen and reactive oxygen species, thereby inhibiting growth and activity of fungi.

9. The product of the contact lens for photodynamic inactivation of the germs of claim 8, wherein the accommodating portion is opaque.

10. The product of the contact lens for photodynamic inactivation of the germs of claim 8, wherein the buffer solution is selected from the group consisting of Tris buffer, HEPES buffer, PBS, glycylglycine buffer, and any combination thereof.

11. The product of the contact lens for photodynamic inactivation of the germs of claim 8, wherein the photoactive solution further comprises a stabilizer.

12. The product of the contact lens for photodynamic inactivation of the germs of claim 8, wherein the white light has a light intensity that is greater than or equal to 0.1 mW/cm² at wavelength of 520 nm.

13. The product of the contact lens for photodynamic inactivation of the germs of claim 8, wherein the fungi are drug-resistant strains.

14. The product of the contact lens for photodynamic inactivation of the germs of claim 8, wherein the fungi are Candida spp., Fusarium spp., and/or Aspergillus spp.

15. A method of treating fungal keratitis by applying a contact lens for photodynamic inactivation of germs, comprising:
submerging a contact lens in a photoactive solution, wherein the contact lens is a hydrogel or a silicone hydrogel, and the photoactive solution comprises 0.01 wt % to 1.0 wt % rose bengal, 0.01 wt % to 1.0 wt % hydrogen peroxide and a buffer solution with a balance;
applying the contact lens on an infected eye; and
exposing the infected eye applied with the contact lens to white light for 0.01 hour to 16 hours, wherein the photoactive solution continuously released by the contact lens produces singlet oxygen and reactive oxygen species, thereby inhibiting growth and activity of fungi, and the photoactive solution excludes an antifungal agent.

16. The method of treating fungal keratitis by applying the contact lens for photodynamic inactivation of the germs of claim 15, wherein the buffer solution is selected from the group consisting of Tris buffer, HEPES buffer, PBS, glycylglycine buffer, and any combination thereof.

17. The method of treating fungal keratitis by applying the contact lens for photodynamic inactivation of the germs of claim 15, wherein the white light has a light intensity that is greater than or equal to 0.1 mW/cm² at a wavelength of 520 nm.

18. The method of treating fungal keratitis by applying the contact lens for photodynamic inactivation of the germs of claim 15, wherein the white light comprises green light with a light dose of 0.01 J/cm² to 200 J/cm², and a wavelength of the green light is 495 nm to 570 nm.

19. The method of treating fungal keratitis by applying the contact lens for photodynamic inactivation of the germs of claim 15, wherein the fungi are drug-resistant strains.

20. The method of treating fungal keratitis by applying the contact lens for photodynamic inactivation of the germs of claim 15, wherein the fungi are Candida spp., Fusarium spp., and/or Aspergillus spp.
